(19) United States
(12) Patent Application Publication
(10) Pub. No.: US 2025/0127751 A1
(43) Pub. Date: Apr. 24, 2025

(54) Traditional Chinese Medicine Compound Preparation for Tumors and Application thereof

(71) Applicant: Institute of Chinese Materia Medica, China Academy of Chinese Medical Sciences, Beijing (CN)

(72) Inventors: Aihua Liang, Beijing (CN); Jingzhuo Tian, Beijing (CN); Chenyue Liu, Beijing (CN)

(21) Appl. No.: 18/916,812

(22) Filed: Oct. 16, 2024

(30) Foreign Application Priority Data
Oct. 18, 2023 (CN) 202311353337.6

(51) Int. Cl. A61K 31/285; A61K 31/404; A61K 31/7076; A61P 35/00

(52) U.S. Cl. CPC A61K 31/285; A61K 31/404; A61K 31/7076; A61P 35/00

(57) ABSTRACT

The disclosure belongs to the technical field of medicine, and specifically discloses a traditional Chinese medicine compound preparation for tumors and application thereof. The traditional Chinese medicine compound preparation includes dimethylarsenic acid, indirubin and cordycepin in a concentration ratio of (1-20):(1-10):(1-40). The traditional Chinese medicine compound preparation of the disclosure can be used for treating kinds of tumors, including leukemia, gastric cancer, lung cancer, glioma, papillary thyroid carcinoma, growth hormone adenoma, pituitary adenoma, myeloma, and other malignant tumors. The traditional Chinese medicine compound preparation has significant treatment effect, high safety, and good development prospects.

### CROSS-REFERENCE TO RELATED APPLICATION

The present application claims priority from Chinese Patent Application No. 202311353337.6 filed on Oct. 18, 2023, the contents of which are incorporated herein by reference in their entirety.

### TECHNICAL FIELD

The disclosure herein relates to the technical field of medicine, and specifically relates to a traditional Chinese medicine compound preparation for tumors and application thereof.

### BACKGROUND

Cell-cycle dysregulation plays an important role in the occurrence of tumors. Cyclin-dependent kinase 2 (CDK2) can form a complex with cyclin and is considered the main rate-limiting factor for the transition from G1 phase to S phase of the cell cycle. Overexpression of CDK2 can accelerate the S phase progress and cause excessive cell proliferation (Cyclin-dependent kinase 2 protects podocytes from apoptosis, Saurus et al, Sci Rep, 2016, 15, doi: 10.1038/srep21664.). P27 is a cyclin-dependent kinase inhibitor. Studies have shown that the p27 protein mainly inhibits kinase complexes such as CyclinE/CDK2. Low expression or loss of expression of p27 in cells may lead to the occurrence of tumors (Study on Cyclin E/CDK2 and p27 in G1/S regulation of gastric cancer cell cycle, Zhang Guidong et al., Journal of Modern Chinese and Western Medicine, 2014, 23 (3): 251-253). Numerous studies have shown that p27 and CDK2 play important roles in the occurrence and development of kinds of tumor diseases such as gastric cancer, lung cancer, glioma, papillary thyroid carcinoma, growth hormone adenoma, pituitary adenoma, and myeloma.

Leukemia is a malignant tumor of the hematopoietic system, divided into acute and chronic types, and according to different cell series, acute leukemia is further divided into acute myeloid leukemia (AML) and acute lymphoid leukemia (ALL). Acute promyelocytic leukemia (APL) is the M3 type of AML and the first malignant tumor targeted tumor specific marker molecules to treat. 95% or more of APL patients have characteristic nonrandom chromosomal translocations, which occur through t (15; 17)-specific chromosomal translocations to form PML-RARA fusion genes, the protein products of which cause cell differentiation arrest and insufficient apoptosis, where APL accounts for approximately 10-15% of AML in the same phase.

The “Chinese Guidelines for the Diagnosis and Treatment of Acute Promyelocytic Leukemia”, Hematology Branch of the Chinese Medical Association, Chinese Journal of Hematology, Volume 39, Issue 3, Pages 179-183, March 2018, states that all-trans-retinoic acid (ATRA)+arsenical is the preferred treatment for APL. The ATRA+arsenical (arsenious acid+compound Huangdai tablets) treatment regimen has significant therapeutic effects, with a five-year disease-free survival rate of 90% or higher. Currently, the ATRA+arsenical treatment regimen has become the first-line treatment for APL in clinical practice. However, the arsenical currently used in clinical practice has varying degrees of safety issues during the treatment process. Especially the arsenical (arsenic trioxide) administered intravenously can cause kinds of adverse reactions such as cardiac toxicity, neurotoxicity, dermal toxicity, liver toxicity, bone marrow transplantation and leukocytosis. Therefore, compared to intravenous administration, oral administration has higher advantages in terms of patient adaptation, drug resistance and cost-effectiveness.

Oral arsenical Qinghuang San contains realgar and indigo naturalis. The main components of indigo naturalis include indirubin (IDB) and indigo, and realgar mainly contains arsenic disulfide. In the study of arsenic metabolites in the body after oral administration of realgar, it is found that single or repeated administration of realgar results in the highest levels of dimethylarsenic acid (DMA) in the blood. There are significant organ differences in the distribution of DMA, with the highest distribution in the blood, the level in the liver and kidneys being about 1/10 of that in the blood, and the level in brain tissue being 1/10 of that in the liver and kidneys (Study of the accumulation and distribution of arsenic species and association with arsenic toxicity in rats after 30 days of oral realgar administration, Yi Y et al, Journal of Ethnopharmacology, 247, 2018, DOI: 10.1016/j.jep.2018.10.037). Compared with trivalent arsenic and pentavalent arsenic, DMA has relatively lower toxicity. The LD₅₀ of arsenic trioxide is 20-50 mg/kg, while the LD₅₀ of DMA is more than 1000 mg/kg (Research progress on different forms and toxicity of arsenic in realgar, Gao Shuangrong et al., Chinese Journal of Experimental Traditional Medical Formulae, Volume 17, Issue 24, Pages 243-247, December 2011). Therefore, it is indicated that compared to arsenic trioxide, DMA is a compound with relatively low toxicity and may have certain prospects in the treatment of APL.

Cordycepin (CDY) is the core component of Cordyceps militaris, and has kinds of pharmacological activities such as anti-inflammation, anti-tumor, and immune regulation. As early as 1997, the United States began using cordycepin in phase I clinical trials for the treatment of acute pre-B and pre-T lymphocytic leukemia, and the experiment has now passed phase II clinical trials (Synergistic property of cordycepin in cultivated Cordyceps militaris-mediated apoptosis in human leukemia cells, Chou S et al, Phytomedicine, 2014, 21 (12): 1516-1524.). Therefore, cordycepin also has certain application prospects in the treatment of leukemia.

It is necessary to develop an oral preparation with relatively high safety and significant therapeutic effects on kinds of tumors, as the safety and therapeutic effects of the prior art cannot be simultaneously considered in the treatment of tumors.

### SUMMARY

To solve the above technical problems, the disclosure provides a traditional Chinese medicine compound preparation for tumors and application thereof. The compound preparation has relatively high safety and potential therapeutic effects on kinds of tumor cells. The compound preparation has good development prospects both in terms of therapeutic effects and drug safety.

To achieve the above objective, the disclosure uses the following technical solution:

On one hand, the disclosure provides a traditional Chinese medicine compound preparation DCI for tumors, including dimethylarsenic acid, indirubin and cordycepin.

Dimethylarsenic acid (DMA) is a highly distributed arsenic metabolite of the traditional Chinese medicine realgar in the body (especially in the blood), indirubin is the main active component in the traditional Chinese medicine indigo naturalis, and cordycepin is the core component of the traditional Chinese medicine Cordyceps militaris.

Preferably, the concentration ratio of the dimethylarsenic acid to the indirubin to the cordycepin is (1-20):(1-10):(1-40).

More preferably, the concentration ratio of the dimethylarsenic acid to the indirubin to the cordycepin is (1-20):(1-10):(1-10).

Most preferably, the concentration ratio of the dimethylarsenic acid to the indirubin to the cordycepin is 1:1:1.

Preferably, the concentration of the dimethylarsenic acid is 10-40 μM, the concentration of the indirubin is 10-40 μM, and the concentration of the cordycepin is 10-40 μM.

More preferably, the concentration of the dimethylarsenic acid is 10-20 μM, the concentration of the indirubin is 10-20 μM, and the concentration of the cordycepin is 10-20 μM.

The disclosure further provides application of the aforementioned traditional Chinese medicine compound preparation in preparation of drugs for inhibiting tumor cell proliferation.

The disclosure further provides application of the aforementioned traditional Chinese medicine compound preparation in preparation of drugs for treating tumors.

Preferably, the tumors include leukemia, gastric cancer, lung cancer, glioma, papillary thyroid carcinoma, growth hormone adenoma, pituitary adenoma, or myeloma.

The traditional Chinese medicine compound preparation DCI described in the disclosure achieves the purpose of treating APL by inhibiting proliferation of NB4 cells, inducing differentiation and apoptosis of NB4 cells, and reducing expression of PML-RARa fusion protein. The mechanism of action of the traditional Chinese medicine compound preparation described in the disclosure in treating leukemia, gastric cancer, lung cancer, glioma, papillary thyroid carcinoma, growth hormone adenoma, pituitary adenoma, myeloma, and the like is as follows: DCI downregulates CDK2 expression, upregulates P27 expression, inhibits cell transition from G1 to S phase, and further inhibits tumor cell proliferation, thereby achieving the purpose of treating various malignant tumors mentioned above.

On the other hand, the disclosure provides a drug for treating tumors, including the aforementioned traditional Chinese medicine compound preparation and a pharmaceutically acceptable carrier.

Preferably, the dosage form of the drug includes tablets, capsules, granules, pastes, oral liquids, pills, or syrups.

Beneficial effects of the disclosure:
(1) The traditional Chinese medicine compound preparation DCI of the disclosure can be used for treating kinds of tumors, including leukemia (mainly APL), gastric cancer, lung cancer, glioma, papillary thyroid carcinoma, growth hormone adenoma, pituitary adenoma, myeloma, and the like.
(2) The traditional Chinese medicine compound preparation DCI described in the disclosure achieves the purpose of treating APL by inhibiting proliferation of NB4 cells, inducing differentiation and apoptosis of NB4 cells, and reducing expression of PML-RARα fusion protein. The mechanism of action of the traditional Chinese medicine compound preparation described in the disclosure in treating leukemia, gastric cancer, lung cancer, glioma, papillary thyroid carcinoma, growth hormone adenoma, pituitary adenoma, myeloma, and the like is as follows: DCI downregulates CDK2 expression, upregulates P27 expression, inhibits cell transition from G1 to S phase, and further inhibits tumor cell proliferation, thereby achieving the purpose of treating kinds of malignant tumors mentioned above.
(3) The traditional Chinese medicine compound preparation DCI of the disclosure is based on the Qinghuang San and contains one of six arsenic metabolites, dimethylarsenic acid (DMA), after realgar administration, as well as indirubin (IDB), the main active component of indigo naturalis, in combination with cordycepin (CDY), the core component of Cordyceps militaris, which has a potential anti-tumor effect. DMA has been found in previous studies to have the highest level among all arsenic metabolites distributed in the blood, and has organ distribution differences (distribution: in blood>in liver or kidneys>in brain). Also, with LD₅₀>1000 mg/kg, DMA is a compound with relatively low toxicity and prospects in treatment of APL. Both IDB and CDY have been reported to have potential therapeutic effects on kinds of tumor cells, with CDY currently passing phase II clinical trials for acute pre-B and pre-T lymphocytic leukemia in the United States, and no significant adverse reactions have been reported. Therefore, the traditional Chinese medicine compound preparation DCI with the relatively low toxicity and anti-tumor effects has good development prospects.

### BRIEF DESCRIPTION OF THE DRAWINGS

FIG. 1 shows the NBT positive rate of NB4 cells treated with DMA, CDY and IDB compounds in different combinations, wherein A of FIG. 1 represents a physiological saline control group, B of FIG. 1 represents an ATO group, C of FIG. 1 represents a DMA group, Dof FIG. 1 represents an IDB group, E of FIG. 1 represents a CDY group, F of FIG. 1 represents a DMA+CDY group, G of FIG. 1 represents a DMA+IDB group, H of FIG. 1 represents a CDY+IDB group, and I of FIG. 1 represents a DCI group.

FIG. 2 shows the morphology of NB4 cells treated with DMA, CDY and IDB compounds in different combinations, wherein A of FIG. 2 represents the physiological saline control group, B of FIG. 2 represents the ATO group, C of FIG. 2 represents the DMA group, D of FIG. 2 represents the IDB group, E of FIG. 2 represents the CDY group, F of FIG. 2 represents the DMA+CDY group, G of FIG. 2 represents the DMA+IDB group, H of FIG. 2 represents the CDY+IDB group, and I of FIG. 2 represents the DCI group.

FIG. 3 shows the degradation of PML-RARα fusion protein in NB4 cells treated with DMA, CDY and IDB compounds in different combinations for 24 h and 96 h, wherein A of FIG. 3 represents the physiological saline control group, B of FIG. 3 represents the ATO group, C of FIG. 3 represents the DMA group, D of FIG. 3 represents the CDY group, E of FIG. 3 represents the IDB group, F of FIG. 3 represents the DMA+CDY group, G of FIG. 3 represents the DMA+IDB group, H of FIG. 3 represents the CDY+IDB group, I of FIG. 3 represents the DCI group, and GAPDH is an internal reference.

FIG. 4 shows the effect on cell cycle regulatory factors in NB4 cells treated with DMA, CDY and IDB compounds in different combinations for 24 h and 96 h, wherein A of FIG. 4 represents the physiological saline control group, B of FIG. 4 represents the ATO group, C of FIG. 4 represents the DMA group, D of FIG. 4 represents the CDY group, E of FIG. 4 represents the IDB group, F of FIG. 4 represents the DMA+CDY group, G of FIG. 4 represents the DMA+IDB group, H of FIG. 4 represents the CDY+IDB group, I of FIG. 4 represents the DCI group, and GAPDH is an internal reference.

FIG. 5 shows the effect of different doses of DMA on the survival time and body weight of NB4-bearing mice in a survival experiment.

FIG. 6 shows the effect of different doses of DMA on the survival time of NB4-bearing mice in the survival experiment, wherein A of FIG. 6 shows the survival time of each group; and B of FIG. 6 shows the survival time lengthening rate of each group, with *p<0.05, ** p<0.01 compared to the control group.

FIG. 7 shows the effect of different doses of DMA on the body weight of NB4-bearing mice in the survival experiment (in a survival state of the control group), with *p<0.05 compared to the control group.

FIG. 8 shows the accumulation of DMA in the blood of rats at the time points of dosing with realgar for 60 d and 90 d, and at the time points of drug withdrawal for 30 d and 60 d, with ¹⁾p<0.05, ²⁾p<0.01, and ³⁾p<0.001 compared to the control group at the same time points.

FIG. 9 shows the accumulation of DMA in the livers of rats at the time points of dosing with realgar for 60 d and 90 d, and at the time points of drug withdrawal for 30 d and 60 d, with ¹⁾p<0.05, ²⁾p<0.01, and ³⁾p<0.001 compared to the control group at the same time points.

FIG. 10 shows the accumulation of DMA in the kidneys of rats at the time points of dosing with realgar for 60 d and 90 d, and at the time points of drug withdrawal for 30 d and 60 d, with ¹⁾p<0.05, ²⁾p<0.01, and ³⁾p<0.001 compared to the control group at the same time points.

FIG. 11 shows the accumulation of DMA in the brain tissue of rats at the time points of dosing with realgar for 60 d and 90 d, and at the time points of drug withdrawal for 30 d and 60 d, with ¹⁾p<0.05, ²⁾p<0.01, and ³⁾p<0.001 compared to the control group at the same time points.

FIG. 12 shows the accumulation of DMA in the urine of rats at the time points of dosing with realgar for 60 d and 90 d, and at the time points of drug withdrawal for 30 d and 60 d, with ¹⁾p<0.05, ²⁾p<0.01, and ³⁾p<0.001 compared to the control group at the same time points.

FIG. 13 shows the pathological images of the liver and kidney tissue of rats after dosing with realgar for 90 d and after drug withdrawal for 60 d, wherein A of FIG. 13: the liver tissue of the control group after dosing for 90 d; B of FIG. 13: the liver tissue of the high-realgar-dose group after dosing for 90 d, the black rectangular box area exhibiting focal mild inflammatory cell infiltration; C of FIG. 13: the liver tissue of the high-realgar-dose group after drug withdrawal for 60 d; D of FIG. 13: the nephridial tissue of the control group after dosing for 90 d; E of FIG. 13: the nephridial tissue of the high-realgar-dose group after dosing for 90 d, the black rectangular box area exhibiting focal mild renal tubular degeneration in the nephridial tissue; and F of FIG. 13: the nephridial tissue of the high-realgar-dose group after drug withdrawal for 60 d; HE stained, 20 μm showed in bar.

### DETAILED DESCRIPTION OF THE EMBODIMENTS

The following examples are only used to help understand the method and core idea of the disclosure. It should be pointed out that for those of ordinary skill in the art, several improvements and modifications can be made to the disclosure without departing from the principles of the disclosure, and these improvements and modifications also fall within the scope of protection as claimed in the disclosure. The following description of the disclosed examples enables those of professional skill in the art to implement or use the disclosure. Various amendments to these examples will be apparent to those of professional skill in the art, and the general principles defined herein can be implemented in other examples without departing from the spirit or scope of the disclosure. Therefore, the disclosure will not be limited to the examples shown herein, but can be applied to a wider range consistent with the principles and novel features disclosed herein. Although any methods and materials similar or equivalent to those described in the disclosure may be used in the implementation or testing of the disclosure, preferred methods and materials are listed here.

Unless otherwise defined, all technical and scientific terms used herein have the same meanings as those commonly understood by those of ordinary skill in the art to which the disclosure belongs.

The following should be noted that:
1. Information of raw materials and instruments used in the experiment is as follows:

Dimethylarsenic acid (DMA): purchased from Ehrenstorfer Standard Company in Germany, dissolved in sterile test water, stored at a concentration of 1 mM (M is mol/L), and kept at −20° C. for later use.

Indirubin (IDB): purchased from China National Institutes for Food and Drug Control, dissolved in dimethyl sulfoxide, filtered through a 0.22 μM filter, stored at a concentration of 5 mM, and kept at −20° C. for later use.

Cordycepin (CDY): also purchased from China National Institutes for Food and Drug Control, dissolved with sterile test water, stored at a concentration of 1 mM, and kept at −20° C. for later use.

RPMI1640 powder and penicillin-streptomycin double antibody: Gibco; fetal bovine serum: Hyclone; microscope: Olympus, Japan; and flow cytometer: Luminex-Guava series. 2. Cell source and cell culture
(1) NB4 cells: also known as human acute promyelocytic leukemia cells, leukemia cells established in 1991 from a female patient with secondary relapse of APL, with characteristic chromosomal translocation t (15:17) and the resulting PML-RARα fusion gene, being ideal in vitro cell models for studying APL, and purchased from BeNa Culture Collection.
(2) Cell culture: cells are seeded at an initial concentration of 5×10⁵ cells/well (3 mL of cell suspension per well in a 6-well plate) into RPMI1640 cell culture medium containing 10% fetal bovine serum and 1% double antibody, and cultured under a 37° C., 5% CO2 saturated humidity condition. At the logarithmic growth phase, a drug is used, and the dosing time is adjusted according to the experimental purpose.

### Example 1 ANNEXIN V-FITC/PI Double-Staining Method for Detecting Cell Apoptosis Rate

The ANNEXIN V-FITC/PI double-staining method is established based on a principle of asymmetric loss of cell membrane in apoptotic cells, that is, externalization of phosphatidylserine. Determination of results: normal live cells: annexin V⁻PI⁻; apoptotic cells: annexin V⁺PI⁻, and necrotic or late apoptotic cells: annexin V⁺PI⁺.

### (1) Experimental Method:

NB4 cells at the logarithmic growth phase were seeded at a density of 1×10⁵ cells/well into a 6-well plate, and dosed 24 h later. The proapoptotic effect of each group was detected according to the dosing methods in Table 1. The cells were collected 24 h after drug dosing and detected on a flow cytometer according to the operating procedure of an ANNEXIN V-FITC/PI apoptosis detection kit (purchased from Solarbio). The experiment was repeated three times. The results are shown in Table 1.

### (2) Experimental Results:

TABLE 1
Effect of each group on apoptosis rate of NB4 cells (X̅ ± SD)

| | | Dose (μM) | | | | Apoptosis rate of |
|---|---|---|---|---|---|---|
| Group | | | DMA | IDB | CDY | NB4 cells (%) |
| Blank | Physiological saline | / | 0 | 0 | 0 | 16.73 ± 5.04 |
| control | control group | | | | | |
| Positive | ATO group | 2 | 0 | 0 | 0 | 47.23 ± 1.70*** |
| control | | | | | | |
| Sample 1 | DMA | / | 20 | 0 | 0 | 30.18 ± 8.75* |
| Sample 2 | IDB | / | 0 | 20 | 0 | 28.88 ± 11.51 |
| Sample 3 | CDY | / | 0 | 0 | 20 | 10.70 ± 3.54 |
| Sample 4 | DMA | / | 40 | 0 | 0 | 33.72 ± 6.86* |
| Sample 5 | IDB | / | 0 | 40 | 0 | 31.38 ± 8.92* |
| Sample 6 | CDY | / | 0 | 0 | 40 | 19.39 ± 5.64 |
| Sample 7 | DMA + IDB | | 15 | 15 | 0 | 31.38 ± 3.96* |
| Sample 8 | DMA + CDY | | 15 | 0 | 15 | 24.30 ± 2.49 |
| Sample 9 | IDB + CDY | | 0 | 15 | 15 | 19.18 ± 0.23 |
| Sample 10 | DMA + IDB | | 30 | 30 | 0 | 37.02 ± 6.39** |
| Sample 11 | DMA + CDY | / | 30 | 0 | 30 | 27.12 ± 2.34* |
| Sample 12 | IDB + CDY | / | 0 | 30 | 30 | 22.09 ± 1.12 |
| Sample 13 | DMA + IDB | / | 40 | 40 | 0 | 39.39 ± 5.59** |
| Sample 14 | DMA + CDY | / | 40 | 0 | 40 | 30.89 ± 3.92* |
| Sample 15 | IDB + CDY | / | 0 | 40 | 40 | 28.35 ± 4.08* |
| Sample 16 | DMA + IDB + CDY | / | 40 | 40 | 40 | 54.40 ± 0.30*** |
| Sample 17 | DMA + IDB + CDY | / | 20 | 20 | 20 | 47.85 ± 0.92*** |
| Sample 18 | DMA + IDB + CDY | / | 10 | 10 | 10 | 40.71 ± 1.98** |
| Sample 19 | DMA + IDB + CDY | / | 5 | 5 | 5 | 32.49 ± 0.77* |
| Sample 20 | DMA + IDB + CDY | / | 20 | 20 | 2 | 40.17 ± 4.30** |
| Sample 21 | DMA + IDB + CDY | / | 40 | 20 | 20 | 45.72 ± 2.58*** |
| Sample 22 | DMA + IDB + CDY | / | 20 | 20 | 80 | 42.18 ± 11.66** |

Note:
*P < 0.05,
**P < 0.01,
***P < 0.001
compared to the physiological saline control group

From the data in the table above, when three compounds were dosed separately, the proapoptotic effects of DMA and IDB at a concentration of 20 μM or higher are significantly higher compared to the control group (p<0.05), indicating a dose-dependent relationship. When all the three compounds were used, the apoptosis rate is the highest, significantly higher than that of DMA used alone, and also significantly higher than that of the three compounds used in pairs, indicating that the use of all the three compounds has the highest proapoptotic effect and none of the three compounds can be absent. The proapoptotic effect is significantly lower in the absence of any of the three compounds, and the three compounds have the most significant proapoptotic effect when proportioned in an equal ratio. For example, sample 16 has a significantly higher proapoptotic effect than sample 22; sample 17 has a significantly higher proapoptotic effect than samples 10, 11 and 12; and sample 18 has a significantly higher proapoptotic effect than samples 7, 8 and 9. However, when the concentration is higher than 20 μM, disruption of a small number of cells was observed under a microscope. As the concentration further increases (>20 μM), the proportion of early apoptosis decreases significantly, while the proportion of late apoptosis increases significantly. Late apoptosis means that a large number of cells will die. Therefore, in subsequent experiments, when the three compounds were used, the recommended concentration is 20 μM for a short experimental time (less than or equal to 24 h), and 10 μM for a relatively long experimental time (more than 48 h). The principle for selecting a positive drug is consistent with that of the test substance, that is, most of the apoptosis observed in the 5 μM group of the positive drug is late apoptosis rather than early apoptosis. Therefore, the dose of the positive drug is regulated as 2 μM for a short experimental time, while for a relatively long experimental time (more than 48 h), the dose of the positive drugs is set as 0.5 μM.

### Example 2 Reduction Experiment of Nitroblue Tetrazolium (NBT)

When normal neutrophils or differentiated cells are stimulated, the production of intracellular superoxide anions increases. NBT receives hydrogen from NADPH in the differentiated cells and reduces into insoluble blue black or blue purple particles—formazane particles. Color spots can be clearly observed under a microscope and the percentage of positive cells can be quantitatively counted to investigate the effect of drug dosing on cell differentiation.

### (1) Experimental Method:

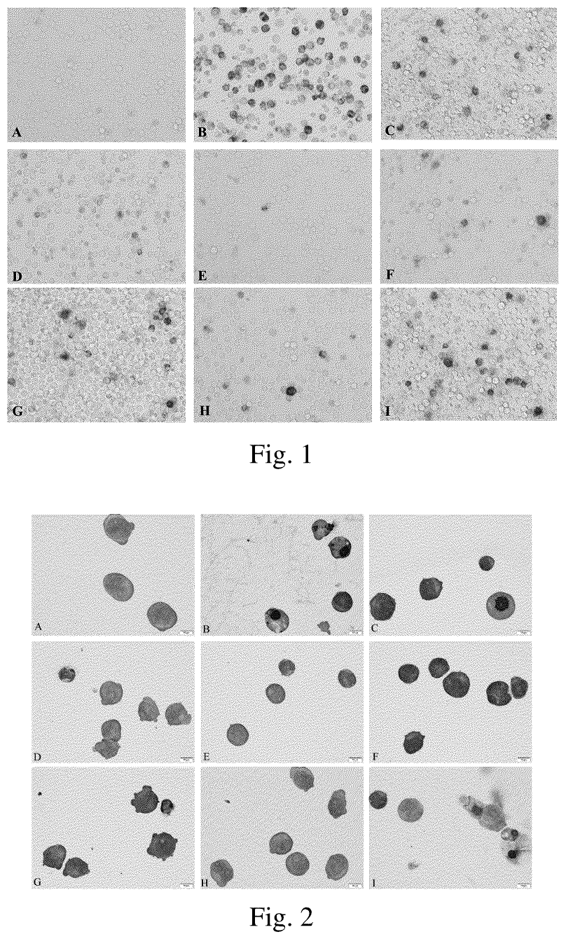

NB4 cells at the logarithmic growth phase were seeded at a density of 2×10⁵ cells/well into a 6-well plate, and dosed 24 h later. According to Table 2, the positive drug ATO, DMA, IDB, CDY, DMA+IDB, DMA+CDY, IDB+CDY, and DMA+CDY+IDB (DCI) were dosed continuously for 10 d, and the cell density per well was controlled at 5×10⁵ cells/well or lower during dosing. 24 h after the last dose, the culture medium was discarded, and the cells were washed twice with PBS. 500 μL of a 0.1% NBT solution (purchased from Sigma and prepared from PBS) containing 1 μg/ml of phorbol ester (PMA, purchased from Sigma) was added to each well, and the cells were incubated at 37° C. for 30 min, washed twice with PBS, and observed under a microscope. In a 400× magnification field, 200 cells were randomly counted from each specimen. Cells containing gray or black formazane particles in the cytoplasm were counted as NBT reduction positive cells, and the percentage of the reduction positive cells was calculated. The experiment was repeated three times. The specific results are shown in Table 2 and FIG. 1.

### (2) Experimental Results:

TABLE 2
Results of NBT reduction positive rate in NB4 cells treated
with DMA, CDY and IDB in different combinations (X ± SD)

| | | | | | NBT reduction |
|---|---|---|---|---|---|
| | Dose (μM) | | | | positive rate |
| Group | | DMA | IDB | CDY | in NB4 cells (%) |
| Physiological saline | / | 0 | 0 | 0 | 0 |
| control group | | | | | |
| ATO group | 0.5 | 0 | 0 | 0 | 57.83 ± 1.98*** |
| DMA | / | 30 | 0 | 0 | 30.24 ± 6.29* |
| IDB | / | 0 | 30 | 0 | 15.20 ± 4.10* |
| CDY | / | 0 | 0 | 30 | 3.19 ± 2.09 |
| DMA + CDY | / | 15 | 0 | 15 | 18.70 ± 1.82* |
| DMA + IDB | / | 15 | 15 | 0 | 36.97 ± 2.26** |
| IDB + CDY | / | 0 | 15 | 15 | 20.70 ± 3.67* |
| DMA + IDB + CDY | / | 10 | 10 | 10 | 49.92 ± 1.90*** |

Note:
*P < 0.05,
**P < 0.01,
***P < 0.001 compared to the physiological saline control group

The morphological and counting results of the cells in the NBT reduction experiment indicate that the NBT reduction rate of the groups dosed with DMA, IDB and CDY alone is 30.24%, 15.20% and 3.19%, respectively. In the pairwise groups, the NBT reduction rate of the DMA+CDY group is 36.97%, which is relatively high, the NBT reduction rate of the DMA+IDB group is 18.70%, and the NBT reduction rate of the IDB+CDY group is 20.70%. Compared with use of the compounds alone or in pairs, the use of all the three compounds (DCI) significantly increases the NBT reducing ability of NB4 cells (P<0.001), with an increase of 49.92%, indicating that the three compounds have the highest promoting effect on cell differentiation. The three compounds show a synergistic effect, and the promoting effect on cell differentiation significantly decreases with the absence of any of the compounds. Moreover, cell debris could be observed under a microscope when the DMA was used alone, indicating that although the DMA alone has a certain promoting effect on cell differentiation, cell damage and disruption can be caused (C in FIG. 1).

### Example 3 Detection of Differentiation Antigen on Cell Membrane Surface

In an immunological marker examination of APL cells, CD11b is negative and CD33 is positive. The surface antigen CD33 is associated with the degree of cellular immaturity, while the surface antigen CD11b is associated with the degree of cellular maturity, High expression of CD11b, as an adhesion molecule on the cell surface, may be a marker of cell differentiation maturation. If the expression of CD33 decreases and the expression of CD11b increases after dosing, it is indicated that the drug may be conducive to inducing cell differentiation.

### (1) Experimental Method:

NB4 cells at the logarithmic growth phase were seeded at a density of 2×10⁵ cells/well into a 6-well plate, and dosed 24 h later. According to doses in Table 3, the positive drug ATO, DMA, IDB, CDY, DMA+IDB, DMA+CDY, IDB+CDY, and DMA+CDY+IDB (DCI) were dosed continuously for 10 d, and the cell density per well was controlled at 5×10⁵ cells/well or lower. 24 h after the last dosing, cells were collected, centrifuged to remove the culture medium, washed twice with precooled PBS, and resuspended in 100 μL PBS. APC (allophycocyanin) and BB515 labeled CD11b and CD33 monoclonal antibodies and corresponding isotype antibodies (both purchased from BD, USA) were added respectively. Following mixing, the cells were incubated at 4° C. for 30 min, filtered through a cell filter membrane, and then detected. The specific results are shown in Tables 3 and 4.

### (2) Experimental Results:

TABLE 3
Statistical results of positive rate of CD11b
expression in NB4 cells treated with DMA, CDY
and IDB in different combinations (X ± SD)

| | | | | | Positive rate of |
|---|---|---|---|---|---|
| | Dose (μM) | | | | CD11b expression |
| Group | | DMA | IDB | CDY | in NB4 cells (%) |
| Physiological saline | / | 0 | 0 | 0 | 0.82 ± 0.23 |
| control group | | | | | |
| ATO group | 0.5 | 0 | 0 | 0 | 42.46 ± 2.66*** |
| DMA | / | 30 | 0 | 0 | 31.90 ± 11.43** |
| IDB | / | 0 | 30 | 0 | 26.42 ± 10.91* |
| CDY | / | 0 | 0 | 30 | 1.79 ± 1.42 |
| DMA + CDY | / | 15 | 0 | 15 | 30.98 ± 14.11* |
| DMA + IDB | / | 15 | 15 | 0 | 39.48 ± 9.80** |
| IDB + CDY | / | 0 | 15 | 15 | 26.72 ± 13.13* |
| DMA + IDB + CDY | / | 10 | 10 | 10 | 45.96 ± 5.43*** |

Note:
*P < 0.05,
**P < 0.01,
***P < 0.001 compared to the physiological saline control group

TABLE 4
Statistical results of positive rate of CD33
expression in NB4 cells treated with DMA, CDY
and IDB in different combinations (X ± SD)

| | | | | | Positive rate of |
|---|---|---|---|---|---|
| | Dose (μM) | | | | CD33 expression |
| Group | | DMA | IDB | CDY | in NB4 cells (%) |
| Physiological saline | / | 0 | 0 | 0 | 40.24 ± 2.11 |
| control group | | | | | |
| ATO group | 0.5 | 0 | 0 | 0 | 18.96 ± 4.63*** |
| DMA | / | 30 | 0 | 0 | 22.06 ± 11.82** |
| IDB | / | 0 | 30 | 0 | 27.50 ± 16.67* |
| CDY | / | 0 | 0 | 30 | 37.14 ± 15.90 |
| DMA + CDY | / | 15 | 0 | 15 | 23.90 ± 13.99* |
| DMA + IDB | / | 15 | 15 | 0 | 20.04 ± 7.08** |
| IDB + CDY | / | 0 | 15 | 15 | 28.22 ± 12.21* |
| DMA + IDB + CDY | / | 10 | 10 | 10 | 17.64 ± 3.99*** |

Note:
*P < 0.05,
**P < 0.01,
***P < 0.001 compared to the physiological saline control group

Results of CD11b: After dosing, the CD11b expression in all groups significantly increases compared to the control group, wherein the DCI group with the three compounds has the highest CD11b expression, with a positive rate of 45.96%, which increases by 5504.88% compared to the control group. It is indicated that the use of all the three compounds shows the highest effect in promoting differentiation and maturation of promyelocytes and myelocytes, which is much higher than the use of the three compounds alone or in pairs.

Results of CD33: After dosing, the CD33 expression in all groups significantly decreases compared to the control group, wherein the DCI group with the three compounds has the lowest CD33 expression, with a positive rate of 17.64%, which decreases by 128.12% compared to the control group. It is indicated that the use of all the three compounds shows the highest effect in promoting differentiation of NB4 cells, which is much higher than the use of the three compounds alone or in pairs.

### Example 4 Cell Morphology Detection Experiment

### (1) Experimental Method:

NB4 cells at the logarithmic growth phase were seeded at a density of 2×10⁵ cells/well into a 6-well plate, and dosed 24 h later. According to doses in Table 2, the positive drug ATO, DMA, IDB, CDY, DMA+IDB, DMA+CDY, IDB+CDY, and DMA+CDY+IDB (DCI) were dosed for culture continuously for 10 d, and the cell density per well was controlled at 5×10⁵ cells/well or lower. NB4 cells cultured for 10 d were centrifuged at 110 g for 3 min, smeared, fixed with methanol, and air dried naturally. Then, the cell smear was stained with Wright's-Giemsa for morphological observation. The experiment was repeated three times. The results are shown in FIG. 2.

### (2) Experimental Results:

After 10 d of intervention with DMA, IDB and CDY alone, a small number of cells in the DMA group showed a decrease in volume and a slight decrease in the nucleus/cytoplasm ratio. Individual cells in the IDB group showed incomplete cell membrane and cell disruption. All cells in the CDY group showed no significant differences from the control group.

In the pairwise groups of the three compounds, a small number of cells in the DMA+CDY group showed a decrease in volume, karyorrhexis into multiple pieces dispersed into the cytoplasm, but relatively intact cell membranes without rupture. A few cells showed a slight decrease in the nucleus/cytoplasm ratio, and an abnormal, irregular kidney-shaped nuclear morphology. Some cells in the DMA+IDB group showed a decrease in volume, an abnormal kidney-shaped nuclear morphology, concentration of a small amount of chromatin in the nucleus, and karyorrhexis into multiple pieces dispersed in the cytoplasm. In addition, a few cells showed incomplete cell membranes and cell disruption. A few cells in the CDY+IDB group showed a slight decrease in the nucleus/cytoplasm ratio, and some cells showed visible vacuoles in the cytoplasm. But the cells showed relatively complete cell membranes and no significant decrease in volume.

Most of cells in the DCI group of the three compounds showed a decrease in volume and obvious nuclear chromatin concentrating, some cells showed an irregular kidney-shaped nuclear morphology and obvious decrease in the nucleus/cytoplasm ratio, and some cells showed visible vacuoles in the cytoplasm, i.e., obvious differentiation. Some cells showed incomplete cell membranes and cell disruption. It is indicated that the use of all the three compounds has the most obvious effect on the cellular morphology and the highest promoting effect on cell differentiation, and the effect obviously decreases when any one of the three compounds is absent.

### Example 5 Western Blot Detection of PML-RARα Protein Expression Level

More than 95% of APL patients have characteristic nonrandom chromosomal translocations t (15:17). The translocation results in fusion of a PML gene on chromosome 15 with an RARα gene on chromosome 17, expressing PML-RARα fusion protein. Research has shown that promoting degradation of the PML-RARα protein can effectively treat APL. Therefore, in the disclosure, the effect of three compounds used in combinations on the expression of the PML-RARα protein was investigated.

### (1) Experimental Method:

NB4 cells at the logarithmic growth phase were seeded at a density of 2×10⁵ cells/well into a 6-well plate, and dosed 24 h later, wherein the cell density per well was controlled at 5×10⁵ cells/well or lower. The positive drug ATO (2 μM for 24 h, and 0.5 μM for 96 h), DMA, IDB, CDY, DMA+IDB, DMA+CDY, IDB+CDY, and DMA+CDY+IDB (DCI) were dosed respectively. The doses of each group for 24 h and 96 h of dosing are shown in the table below.

TABLE 5
Doses of each group

| | Doses (μM) of each group | | | Doses (μM) of each group | | |
|---|---|---|---|---|---|---|
| | for 24 h of dosing | | | for 96 h of dosing | | |
| Group | DMA | IDB | CDY | DMA | IDB | CDY |
| DMA | 60 | 0 | 0 | 30 | 0 | 0 |
| IDB | 0 | 60 | 0 | 0 | 30 | 0 |
| CDY | 0 | 0 | 60 | 0 | 0 | 30 |
| DMA + CDY | 30 | 0 | 30 | 15 | 0 | 15 |
| DMA + IDB | 30 | 30 | 0 | 15 | 15 | 0 |
| IDB + CDY | 0 | 30 | 30 | 0 | 15 | 15 |
| DMA + IDB + CDY | 20 | 20 | 20 | 10 | 10 | 10 |

At the end of the experiment, cells were collected and washed twice with precooled PBS. 200 μL of a protein lysis buffer (RIPA, containing 1% PMSF and 1% phosphatase inhibitor) was added to each well, gently mixed evenly, and allowed to stand for 5 min to observe for precipitation. The cells were centrifuged at 10,000 rpm for 5 min, and the supernatant was taken for detecting the protein concentration using a BCA protein detection kit. A loading buffer was added to a sample with an adjusted concentration, and the sample was boiled in boiling water for 7 min, aliquoted and stored at −20° C. or directly detected.

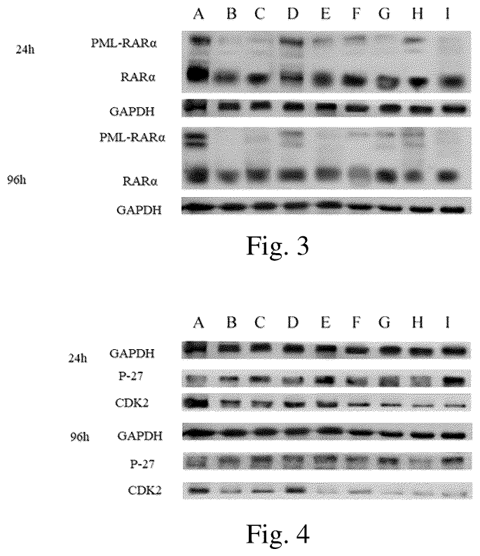

An appropriate gel concentration was selected according to different molecular weight of the protein. The protein was isolated by polyacrylamide gel electrophoresis. First 80 V voltage was applied for isolation, and then 120 V voltage was applied after bromophenol blue reached the separation gel. The strips in the gel were transferred to a 0.22 μm PVDF membrane by wet transfer, blocked with 5% skimmed milk powder or bovine serum albumin BSA-PBST for 2 h, and then blocked overnight with primary antibodies, wherein the primary antibodies were mouse monoclonal antibodies of PML and RARα (Santa Cruz Biotechnology, USA). The primary antibodies were discarded after incubation overnight at 4° C., and the membrane was washed three times with PBST for 10 min each time, incubated with horseradish peroxidase labeled goat anti-mouse secondary antibodies (proteintech) at room temperature for 1.5 h, and then washed three times with PBST for 10 min each time. Finally, an ECL color development solution was added for development using a Western blot imaging system (Protein-sample Biotech, USA). The results are shown in FIG. 3.

### (2) Experimental Results:

Except the group dosed with 20 μM CDY for 24 h, the protein expression of the PML-RARα dosed with the three compounds alone, in pairs and totally showed varying degrees of obvious decrease, wherein the decrease in the protein expression of the DCI group is the most obvious, and is substantially close to the decreasing degree of the positive drug ATO group, with the substantially consistent results in 24 h and 96 h of dosing. It is indicated that the use of the three compounds has the highest degree of promoting protein degradation of PML-RARa, and the promoting effect on protein degradation significantly decreases in the absence of any one of the compounds.

### Example 6 Western Blot Detection of Protein Expression of Key Cell Cycle Regulatory Factors

CDK2 is considered the main rate-limiting factor for the transition from G1 phase to S phase of the cell cycle. Overexpression of CDK2 can accelerate the S phase progress and cause excessive cell proliferation. P27 is an inhibitor of CDK, and expression of P27 can inhibit the progress from G1 to S. If the expression of p27 in NB4 cells is upregulated and the expression of CDK2 is downregulated after dosing, cells can be inhibited from entering the S phase.

### (1) Experimental Method:

NB4 cells at the logarithmic growth phase were seeded at a density of 2×10⁵ cells/well into a 6-well plate, and dosed 24 h later, wherein the cell density per well was controlled at 5×10⁵ cells/well or lower. The positive drug ATO (2 μM for 24 h, and 0.5 μM for 96 h), DMA, IDB, CDY, DMA+IDB, DMA+CDY, IDB+CDY, and DMA+CDY+IDB (DCI) were dosed respectively. The doses are shown in Table 5. The Western blot procedure was substantially the same as the PML-RARα protein expression detection in Example 5, except that the primary antibodies were replaced with mouse monoclonal antibodies of CDK2 and p27 (CST Biotechnology, USA). Upregulation or downregulation in expression of both the CDK2 and p27 can affect the cell cycle and inhibit the progress from G1 to S phase. The results are shown in FIG. 4.

### (2) Experimental Results:

The results of both 24 h and 96 h of dosing showed that, except the CDY group, treatment with the three compounds used alone, in pairs or totally can downregulate the protein expression of the CDK2 and upregulate the protein expression of the P27 to varying degrees. The DCI group has the most significant protein change, and an inhibitory effect significantly higher than that of the groups dosed with the three compounds alone or in pairs. It is indicated that the use of all the three compounds has a synergistic/cumulative effect in inducing G0/G1 phase arrest in NB4 cells, and the absence of any of the three compounds significantly decreases the effect on protein expression.

The above is a further description of the disclosure based on the specific examples. However, the examples are only exemplary and do not limit the scope of the disclosure in any way. Those skilled in the art should understand that the details and forms of the technical solution of the disclosure can be modified or replaced without departing from the spirit and scope of the disclosure, and all the modifications and replacements fall within the scope of protection of the disclosure.

### Example 7 Effect of Intraperitoneal Administration of DMA on Body Weight and Survival Time of Tumor-Bearing Mice

DMA belongs to group 2B carcinogens, indicating a possible carcinogenic effect and posing certain safety risks. In view of this, a short-term toxicity experiment was designed to investigate the effect of DMA on the survival time of tumor-bearing mice, and to monitor the safety thereof while assessing the efficacy thereof.

### (1) Experimental Method:

NB4 cells at the logarithmic growth phase were centrifuged and resuspended in a sterile phosphate buffer and the cell concentration was adjusted to 8×10⁷/mL. Each mouse was intraperitoneally inoculated with 0.2 mL of the liquid containing tumor cells, i.e., 1.6×10⁷ NB4 cells per mouse. After 24 h, the experiment was performed in groups for treatment. SCID mice were divided into 4 groups: a control group and DMA groups (50, 10 and 5 mg/kg respectively), with 10 mice in each group. DMA was dissolved in a 0.9% sodium chloride solution and injected intraperitoneally once every other day for a total of 7 doses. The control group was injected intraperitoneally with the 0.9% sodium chloride solution at a dose by volume of 0.1 ml/10 g.

### (2) Experimental Results:

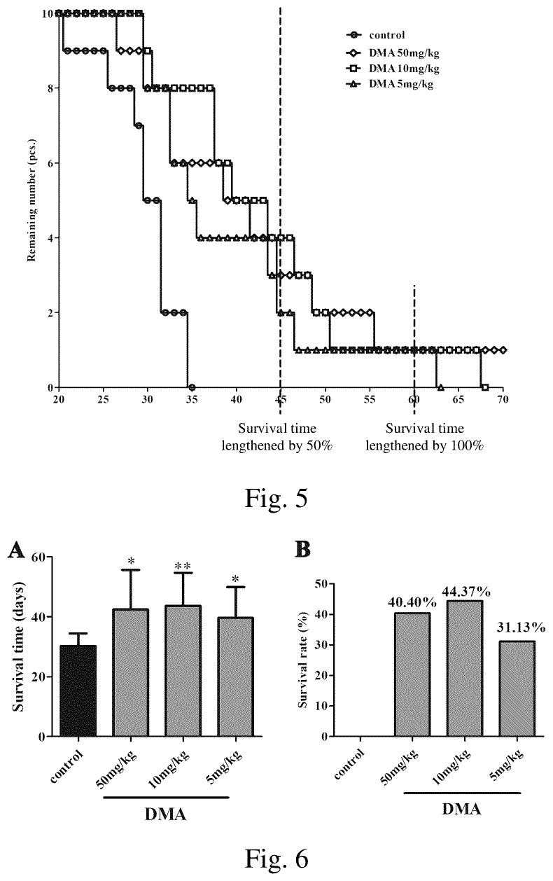

The mean survival time of the control group is 20-35 d (30.2±4.2 d), and the survival time significantly lengthens in the groups receiving high, medium and low doses of DMA compared to the control group (p<0.05). The survival time of the DMA group (50 mg/kg) is 42.4±13.2 d, the survival time of the DMA group (10 mg/kg) is 43.6±11.1 d, and the survival time of the DMA group (5 mg/kg) is 39.6±10.3 d. Compared to the control group, the survival time lengthening rate of each group with different doses of DMA is 40.40% (50 mg/kg), 44.37% (10 mg/kg) and 31.13% (5 mg/kg) respectively. The results showed that the DMA can significantly lengthen the survival time of the tumor-bearing mice, that is, the DMA has a certain therapeutic effect on ascites tumors in NB4-bearing mice. The specific results are shown in FIG. 5 and FIG. 6.

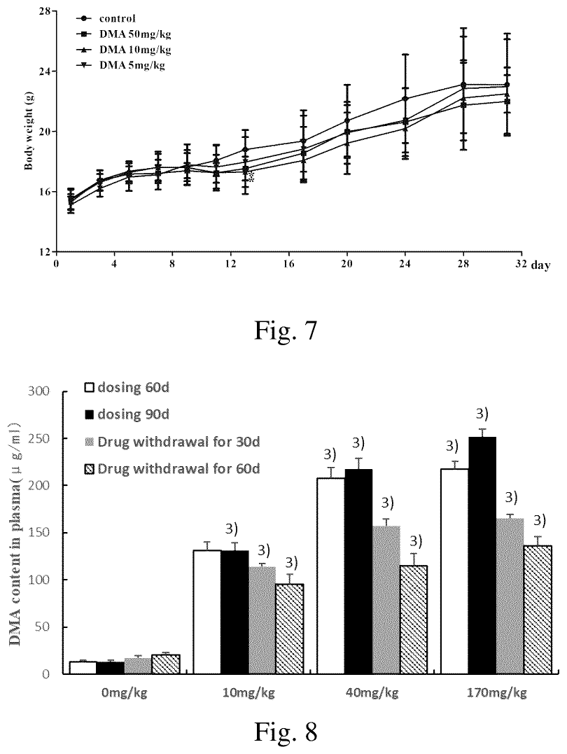

In addition, following administration of the DMA during the experiment, except for a significant decrease in the body weight in the DMA groups (50 and 10 mg/kg) compared to the control group on day 13, there was no significant difference in the body weight between the DMA groups with different doses and the control group at other time points. Therefore, we believe that there were no significant toxic effects caused by administration of the DMA during the experiment. The specific results are shown in FIG. 7.

### Example 8 Study on Accumulation and Carcinogenic Effect of DMA in the Body Caused by Long-Term Administration of Realgar

Previous studies have found that long-term administration of realgar can lead to arsenic accumulation in blood, liver, kidney, and brain tissues, and the toxic effect on the liver and kidney may be related to the arsenic accumulation. Among arsenic metabolites, the accumulation of DMA in tissues and organs is relatively high. Given that DMA belongs to Group 2B carcinogens, long-term administration of realgar may pose a certain carcinogenic risk. Therefore, a subchronic toxicity experiment for realgar was designed to detect the accumulation level of DMA in blood, liver, kidney, and brain tissues, and to investigate whether DMA at a high concentration in the body has a carcinogenic effect.

### (1) Experimental Method:

According to ICH M3 subchronic toxicity experimental guidelines, the subchronic toxicity study of realgar was conducted in a manner of dosing for 90 d and drug withdrawal for 60 d. The experimental animals were male Wistar rats, nearly 7 weeks old. By conversion according to the dosage recorded in the 2020 version «Pharmacopoeia of the People's Republic of China», the highest clinical equivalent dose of realgar is 1.7 mg/kg/day. The doses 10 mg/kg (6 times the equivalent dose), 40 mg/kg (24 times the equivalent dose), and 170 mg/kg (100 times the equivalent dose) were selected for the experiment. Realgar was dissolved in a 0.3% CMC-Na solution to prepare solutions with concentrations of 2 mg/ml, 8 mg/ml, and 34 mg/ml and used immediately. The solutions were orally administered by gavage at a dose by volume of 5 ml/kg daily, and the control group was orally administered with the 0.3% CMC Na solution during the administration process. The status of the animals after daily administration was observed and recorded, and any abnormal deaths were promptly recorded.

Samples were collected at the time points of dosing for 60 d and 90 d as well as drug withdrawal for 30 d and 60 d. The animals were fasted for 16 h before being euthanized, and urine was collected at the same time. The animals were anesthetized to collect blood from the abdominal aorta. The main organs of the animals, including the heart, liver, spleen, lung, kidney, brain, gonad, and the like, were extracted and the organ index was calculated as follows: organ index (average organ weight %)=[organ weight (g)/fasting body weight (g)]×100%. The liver and kidney tissues of the dosed groups were fixed, dehydrated, embedded, and sliced for HE staining to observe whether tumors were induced to evaluate the carcinogenic effect of realgar.

In addition, the content of DMA in blood, liver, kidney, brain, and urine was detected by HPLC-ICP-MS technology. First, a DMA standard stock solution was prepared with pure water and diluted to a working solution on the day of testing. A sample was acidified with hydrochloric acid and eluted on an IEX chromatographic column to obtain the eluent. Considering that DMA can be adsorbed onto the column, pure water was added for separation, and a dissociation solution was collected to obtain the DMA. The content of DMA in a tissue sample was detected by ICP-MS under the following conditions: RF power: 1500 W; sampling depth: 8.0 mm; carrier gas flow rate: 0.9 L/min; and compensation air flow rate for carrier gas: 0.3 L/min. Quantitative analysis (model) was performed for an integration time of 2 s and repeated three times.

### (2) Experimental Results:

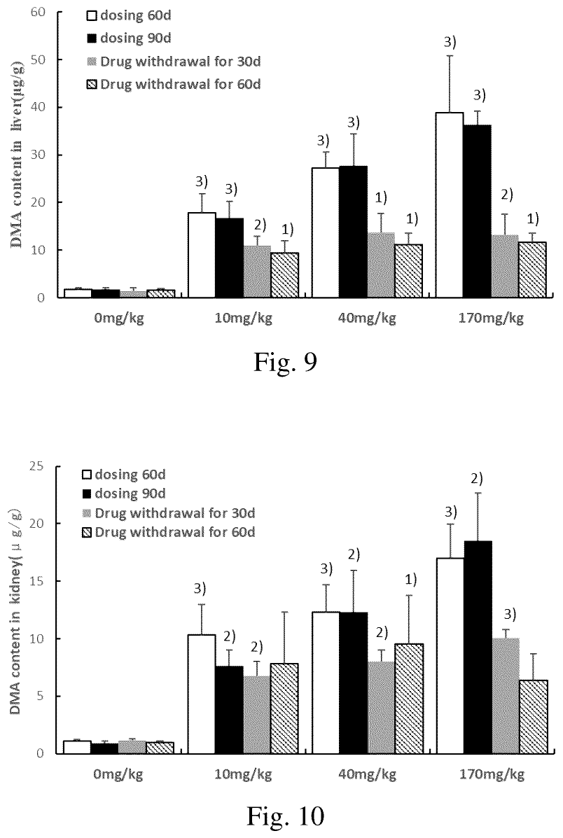

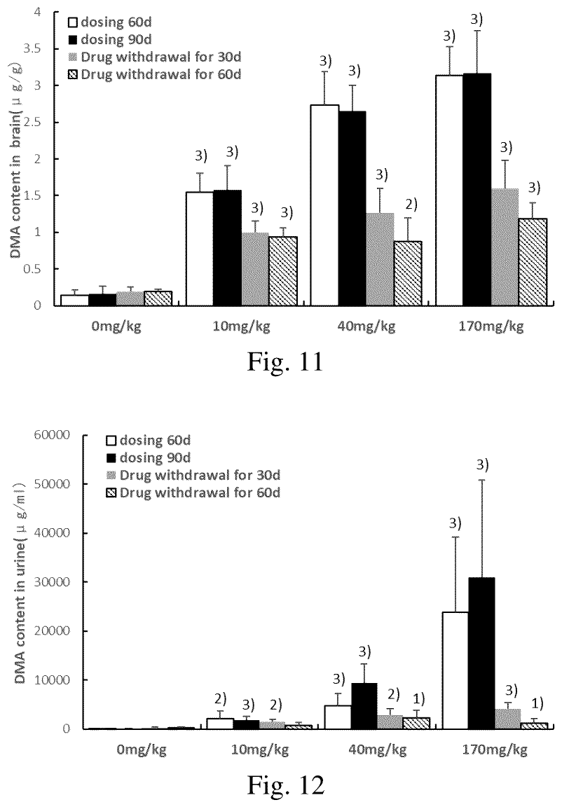

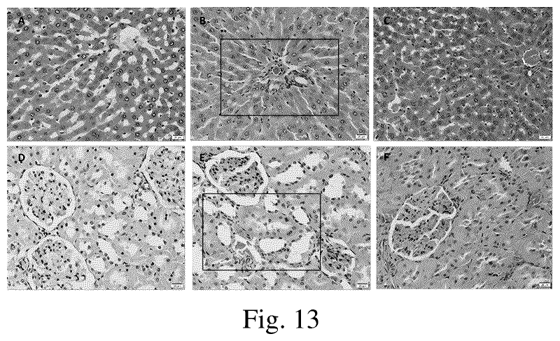

The accumulation of DMA in the blood, urine, liver, kidney, and brain tissues increased significantly with the dosing time. Although the accumulation of DMA in kinds of tissues decreased significantly after drug withdrawal, the content was still significantly higher than that of the control group. After dosing for 90 d, the DMA content in blood of the low-, medium- and high-dose groups was 11.11, 19.93 and 23.65 times that of the control group respectively; the DMA content in liver of the low-, medium- and high-dose groups was 12.58, 17.75 and 28.79 times that of the control group respectively; the DMA content in kidney of the low-, medium- and high-dose groups was 11.01, 15.98 and 22.84 times that of the control group respectively; the DMA content in brain tissue of the low-, medium- and high-dose groups was 9.66, 16.29 and 19.47 times that of the control group respectively; and the DMA content in urine of the low-, medium- and high-dose groups was 16.07, 91.86 and 468.74 times that of the control group respectively, indicating a clear dose-dependent relationship in DMA accumulation. The above indicates that long-term administration of realgar can significantly increase the concentration and accumulation of DMA in major organs such as the blood, liver, kidney, and brain. The specific results are shown in FIG. 8 to FIG. 12.

No animal deaths were observed during the administration process. After dosing for 90 d, there was no significant difference in the organ indices such as heart, liver, spleen, lung, kidney, brain, adrenal gland, seminal vesicle, testis, salivary gland, and prostate indices between the groups with different doses of realgar and the control group. The specific results are shown in Table 6. Pathological results showed that after dosing for 90 d, focal mild inflammatory cell infiltration was observed in the liver tissue of the high-realgar-dose group (100 times the clinical equivalent dose), and focal mild renal tubular degeneration and a small number of exposed renal epithelial cells were observed in the kidney tissue. These pathological changes were local and mild, and no serious pathological changes were observed. Furthermore, similar pathological changes were observed in the liver and kidney tissues of the control group at the same time point, indicating that the pathological changes may be related to the self-state of the animals after long-term feeding and may not be caused by administration. Also, no tumor growth was observed in the liver and kidney tissues. The pathological results of the liver and kidney are shown in FIG. 13.

Compared to the control group, no significant abnormal changes were observed in the brain, lung, bronchi, adrenal gland, pancreas, bladder, gonad and other organs in the high-realgar-dose group. In addition, no significant abnormal pathological changes were observed in the organs of the medium- and low-realgar-dose group. It is indicated that at a over high dose (100 times the clinical equivalent dose), realgar does not cause significant hepatorenal toxicity related to administration, nor does realgar have a significant carcinogenic effect.

TABLE 6
Organ indices of rats orally administered with realgar for 90 d (X ± SD)

| | | Realgar | | |
|---|---|---|---|---|
| | Control group | 10 mg/kg | 40 mg/kg | 170 mg/kg |
| Cardiac index | 0.273 ± 0.024 | 0.260 ± 0.019 | 0.287 ± 0.013 | 0.271 ± 0.007 |
| Liver index | 2.320 ± 0.162 | 2.528 ± 0.154 | 2.520 ± 0.291 | 2.655 ± 0.305 |
| Spleen index | 0.184 ± 0.016 | 0.204 ± 0.009 | 0.208 ± 0.037 | 0.205 ± 0.028 |
| Lung index | 0.342 ± 0.021 | 0.370 ± 0.030 | 0.351 ± 0.035 | 0.340 ± 0.018 |
| Renal index | 0.579 ± 0.039 | 0.597 ± 0.045 | 0.573 ± 0.142 | 0.613 ± 0.082 |
| Brain index | 0.431 ± 0.025 | 0.324 ± 0.175 | 0.394 ± 0.052 | 0.408 ± 0.020 |
| Adrenal gland | 0.039 ± 0.054 | 0.016 ± 0.002 | 0.017 ± 0.001 | 0.017 ± 0.007 |
| index | | | | |
| Seminal vesicle | 0.340 ± 0.063 | 0.379 ± 0.084 | 0.364 ± 0.074 | 0.306 ± 0.031 |
| index | | | | |
| Testis index | 0.766 ± 0.057 | 0.619 ± 0.227 | 0.690 ± 0.119 | 0.711 ± 0.077 |
| Salivary gland | 0.171 ± 0.036 | 0.173 ± 0.032 | 0.198 ± 0.058 | 0.164 ± 0.023 |
| index | | | | |
| Prostate index | 0.226 ± 0.079 | 0.190 ± 0.075 | 0.146 ± 0.013 | 0.167 ± 0.023 |

Based on the safety studies in Examples 7 and 8, it can be concluded that no significant hepatorenal toxicity or carcinogenic effects were observed in administration of DMA during the experimental process of the disclosure.

What is claimed is:

1. A traditional Chinese medicine compound preparation for tumors, comprising dimethylarsenic acid, indirubin and cordycepin.

2. The traditional Chinese medicine compound preparation according to claim 1, wherein the concentration ratio of the dimethylarsenic acid to the indirubin to the cordycepin is (1-20): (1-10): (1-40).

3. The traditional Chinese medicine compound preparation according to claim 1, wherein the concentration ratio of the dimethylarsenic acid to the indirubin to the cordycepin is (1-20): (1-10): (1-10).

4. The traditional Chinese medicine compound preparation according to claim 1, wherein the concentration of the dimethylarsenic acid is 10-40 M, the concentration of the indirubin is 10-40 μM, and the concentration of the cordycepin is 10-40 μM.

5. The traditional Chinese medicine compound preparation according to claim 4, wherein the concentration of the dimethylarsenic acid is 10-20 μM, the concentration of the indirubin is 10-20 μM, and the concentration of the cordycepin is 10-20 μM.

6. Application of the traditional Chinese medicine compound preparation according to claim 1 in preparation of drugs for inhibiting tumor cell proliferation.

7. Application of the traditional Chinese medicine compound preparation according to claim 1 in preparation of drugs for treating tumors.

8. The application according to claim 6, wherein the tumors comprise leukemia, gastric cancer, lung cancer, glioma, papillary thyroid carcinoma, growth hormone adenoma, pituitary adenoma, or myeloma.

9. A drug for treating tumors, comprising the traditional Chinese medicine compound preparation according to claim 1 and a pharmaceutically acceptable carrier.

10. The drug according to claim 9, wherein the dosage form of the drug comprises tablets, capsules, granules, pastes, oral liquids, pills, or syrups.
